(19) United States
(12) Patent Application Publication
(10) Pub. No.: US 2006/0004050 A1
(43) Pub. Date: Jan. 5, 2006

(54) Compositions and methods for the prevention or treatment of pain and other nervous system disorders

(76) Inventors: Brian T. Speicher, Lansdale, PA (US); Robert F. Kucharik, Glenmoore, PA (US)

Correspondence Address: BRIAN SPEICHER, 214 OLD FARM LANE, LANSDALE, PA, 19446, US

(21) Appl. No.: 11/172,269

(22) Filed: Jun. 30, 2005

Related U.S. Application Data
(60) Provisional application No. 60/585,466, filed on Jul. 2, 2004.

(51) Int. Cl. A61K 31/445

(52) U.S. Cl. 514/317

(57) ABSTRACT

A tolperisone-related compound or a compound of Formula Z is administered for the prevention and treatment of periodic paralyses and myotonias of several types, long QT syndrome, Brugada syndrome, malignant hyperthermia, myasthenia, epilepsy, ataxia, migraine, Alzheimer's Disease, Parkinson's Disease, schizophrenia, and hyperekplexia, neuropathic pain, and pain associated with nervous system disorders including, but not limited to, painful diabetic neuropathy, postherpetic neuralgia, trigeminal neuralgia, complex regional pain syndrome, Guillain-Barre syndrome (GBS), Charcot-Marie-Tooth (CMT) disease, complex regional pain syndrome, type 1 (CRPS-1), ischemic neuropathy, fibromyalgia, chronic fatigue syndrome, painful spasticities, and other nervous system disorders that have pain as an attendant sign and/or symptom.

This invention claims priority in part from U.S. Patent Application No. 60/585,466, filed Jul. 2, 2004.

### FIELD OF THE INVENTION

The invention relates to compositions and methods for the prevention or treatment of disorders that arise from altered cell membrane excitability, including neuropathic pain, and pain associated with other nervous system disorders. In particular, the invention relates to the use of pharmaceutical compositions comprising tolperisone-related compounds (including racemic tolperisone, (+)-tolperisone substantially free of (−)-tolperisone, or (−)-tolperisone substantially free of (+)-tolperisone), for the prevention and treatment of disorders that arise from altered cell membrane excitability, including neuropathic pain, and pain associated with other nervous system disorders.

### BACKGROUND OF THE INVENTION

Cell membranes represent a complex system of excitatory and inhibitory checks and balances. See Ptacek (Curr Opin Neurol 1998 November; (11) 217-226) for a detailed review. Examples of inhibitory influences include voltage-gated potassium channels and chloride channels. Excitatory influences include glutamate receptors, voltage-gated sodium channels, and calcium channels. In addition to these primary determinants of membrane excitability, environmental influences such as temperature or exposure to exogenous compounds (caffeine, alcohol and drugs) can also modulate membrane excitability.

The normal range for resting membrane excitability is maintained by a balance of excitatory and inhibitory influences. Despite being tightly controlled, there still may be biologically meaningful variation. Genetic mutations can lead to mutant ion channels that yield low or high net excitability, whose function remains compatible with life, but which gives rise to pathophysiology and disease. There are a wide variety of ion channel related diseases, also termed “channelopathies”, which impart electrophysiological instability and associated dysfunction. Dworakowska and Dolowy (Acta Bio Pol 2000; 47(3): 685-703) as well as Davies and Hanna (Curr Opin Neurol 2003; 16:559-568) provide summaries of these disease states. Clinical diagnoses include (but are not limited to) periodic paralyses and myotonias of several types, long QT syndrome, Brugada syndrome, malignant hyperthermia, myasthenia, epilepsy, ataxia, migraine, Alzheimer's Disease, Parkinson's Disease, schizophrenia, and hyperekplexia.

Physical insult to otherwise healthy cell membrane systems may also give rise to pathology. As discussed by Summer (Curr Opin Neurol. 2003 October; 16(5):623-8) and Krarup (Curr Opin Neurol. 2003 October; 16(5):603-12), injuries to nerve tissue produces a large variety of cellular changes and subsequent pathophysiology, producing electrophysiological instability in cell membranes. The notable feature of neuropathic pain, in contrast to nociceptive pain, is the perturbation of pain signaling pathways resulting from said instability. Insult to the central and peripheral nervous system can result in heightened sensitivity non-noxious stimuli, and/or an exaggerated response to mild to moderate noxious stimuli. As described in Dworkin et al (Arch Neurol 2003 November; 60:1524-34) a simple focal peripheral nerve injury unleashes a range of peripheral and central nervous system processes that can all contribute to persistent pain and abnormal sensation. Inflammation, reparatory mechanisms of neural tissues in response to injury, and the reaction of adjacent tissues to injury lead to a state of hyperexcitability in primary afferent nociceptors, a phenomenon termed peripheral sensitization. Central neurons innervated by such nociceptors undergo dramatic functional changes including a state of hyperexcitability termed central sensitization. Normally these sensitization phenomenon extinguish themselves as the tissue heals and inflammation subsides. However, when primary afferent function is altered in an enduring way by injury or disease of the nervous system, these processes persist and may be highly resistant to treatment.

As reviewed in Bonica's Management of Pain, 3^{rd} Edition, with the sensitization of the peripheral and central nervous system established, the manifestation of neuropathic pain can take on a constellation of positive and negative symptoms. Positive sensory phenomenon relate to the exaggerated perception of stimuli (allodynia, hyperalgesia, hyperpathia), where the application of a modest mechano-thermal challenge results in the false perception of a disproportionate increase of the challenge. Positive motor symptoms include increased muscle tone, tremor, dystonia, and dyskinesiae. Negative sensory phenomena include an inappropriate response to light touch, vibration, joint position, pin prick, or warm/cold application to the affected region. Negative motor symptoms include hypotonia, decreased muscle strength, and decreased endurance. The particular constellation of positive and negative signs and symptoms are often ground in the specific insult to the nervous system.

As reviewed in Carter et al (Physical Medicine and Rehabilitation Clinics of North America 2001 May; 12(2):447-59), neuropathic pain is a common symptom associated with a variety of peripheral nerve disorders, including but not limited to neuropathy associated with diabetes (DN), alcohol, hypothyroidism, uremia, nutritional deficiencies, and chemotherapy (primarily vincristine, cis-platin, zalcitabine, and paclitaxel). Other acquired and inherited disorders, including Guillain-Barre syndrome (GBS), postherpetic neuralgia (PHN), Charcot-Marie-Tooth (CMT) disease, complex regional pain syndrome, type 1 (CRPS-1), and ischemic neuropathy, also may be associated with neuropathic pain.

Hennings has reviewed the impact of sodium channel modulation (Neurosurg Anesthesiol 2004; 16(1); 100-101) and its role in neuroprotection. Sodium channels play a critical part in the generation of interneuronal action potential. In models of neuropathology such as ischemic insult, altered ionic function is evident. See Fried et al, J Physiol 1995; 489:557-565 for a specific discussion. In example, with an ischemic challenge to nervous tissue, neurons rapidly depolarize as ATP synthesis declines. Affected sodium channels lose vital capacity, and the ability to self-regulate. Voltage gated ion channels, including sodium and calcium channels, are activated. Intracellular sodium and calcium increase, prompting the release of chemical signals such as glutamate. The accumulation of glutamate then creates an excitotoxic environment, rendering surrounding neuronal and related tissue vulnerable.

There are also a number of nervous system disorders with attendant pain, such as chronic fatigue syndrome and fibromyalgia. Chronic fatigue syndrome (CFS) is a disorder characterized by fatigue of an incapacitating nature lasting at least six (6) months. Symptoms of CFS include, but are not limited to, mild fever or chills, sore throats, painful lymph nodes, unexplained general muscle weakness, myalgias, prolonged generalized fatigue after exercise previously tolerated, generalized headaches, migratory arthralgias, neuropsychotic complaints, sleep disturbance, and description of a main symptom complex developing over a few hours to a few days.

Fibromyalgia is a disorder related to chronic fatigue syndrome. However, in contrast to chronic fatigue syndrome, major symptoms of fibromyalgia do not include fatigue. Instead, fibromyalgia is characterized by general aches or stiffness involving three or more anatomical sites for at least 3 months and at least six typical and reproducible tender points. Minor symptoms of fibromyalgia include irritable bowel syndrome, and modulation of symptoms by activity, weather and stress. Despite the differences in their definitions, patients with either fibromyalgia or chronic fatigue syndrome share many symptoms and epidemiologic factors.

The prescribing of several drug classes has been attempted with the intention of treating neuropathic pain, pain associated with nervous system disorders, and altered cell membrane excitability. Given the wide array of pathology, the success of each treatment has been very limited. The tolerability (within therapeutically relevant dose ranges) of these drug classes can differ greatly as well. In example, for a review of analgesic treatment modalities for neuropathic pain states, see Bonica's Management of Pain, 3^{rd} Ed. and Dworkin et al (Arch Neurol 2003 November; 60:1524-34).

There is a need for novel treatments that target diseases that arise from altered cell membrane excitability, including neuropathic pain, and pain attendant to nervous system disorders. Potential treatments need to be both tolerable and efficacious. Moreover, given the improved understanding of how acute pain may evolve into chronic and neuropathic pain, there is a need for treatments that are broadly effective in preventing the development of pain from neuropathy or nervous system disorders. Treating a neuropathic pain state or pain from nervous system disorders once it is established is of great importance as well. Such treatment could potentially address the various origins and physical manifestations altered cell membrane excitability, including that of pain. These conditions include, but not limited to, periodic paralyses and myotonias of several types, long QT syndrome, Brugada syndrome, malignant hyperthermia, myasthenia, epilepsy, ataxia, migraine, Alzheimer's Disease, Parkinson's Disease, schizophrenia, and hyperekplexia, painful diabetic neuropathy, postherpetic neuralgia, trigeminal neuralgia, complex regional pain syndrome, Guillain-Barre syndrome (GBS), Charcot-Marie-Tooth (CMT) disease, complex regional pain syndrome, type 1 (CRPS-1), ischemic neuropathy, fibromyalgia, chronic fatigue syndrome, painful spasticities, and other nervous system disorders that have pain as an attendant sign and/or symptom. Potential treatments need to be tolerable to the extent that they would be utilized over extended periods of time.

### SUMMARY OF THE INVENTION

One aspect of the invention is a method for preventing or treating disorders that arise from altered cell membrane excitability, including neuropathic pain, and pain associated with other nervous system disorders in a subject in need of such treatment. The method comprises administering an effective amount of a pharmaceutical composition comprising a tolperisone-related compound and a pharmaceutically acceptable carrier to said subject.

A second aspect of the invention is a method for preventing or treating disorders that arise from altered cell membrane excitability, including neuropathic pain, and pain associated with other nervous system disorders in a subject in need of such treatment which comprises administering an effective amount of a pharmaceutical composition comprising a compound of Formula Z and a pharmaceutically acceptable carrier to said subject. The compound includes one or more R1 or R2 or R3 moieties comprising s a hydride, a halogen, a hydroxyl, a lower alkoxy, lower alkyl, lower alkenyloxy, amino, lower-alkanoyl amino or lower alkylthio group, and the ring-structures contain carbon, amine or oxygen groups and have 4, 5, 6 or 7 carbon skeletons with single or double bonds and n=1-4.

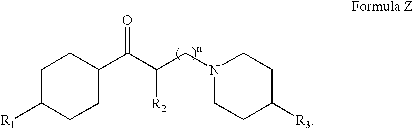

The pharmaceutical compositions may be administered orally, parenterally, or by a transdermal route and in an unmodified or modified release form.

Another aspect of the invention is a pharmaceutical composition comprising either (+)-tolperisone, substantially free of the corresponding (−)-isomer, and a pharmaceutically effective carrier or (−)-tolperisone, substantially free of the corresponding (+)-isomer, and a pharmaceutically effective carrier.

A further aspect of the invention is a pharmaceutical composition comprising high doses of magnesium (about 2 to about 12 times the recommended daily allowance) and either racemic tolperisone, or (+)-tolperisone, substantially free of the corresponding (−)-isomer, or (−)-tolperisone, substantially free of the corresponding (+)-isomer, and a pharmaceutically effective carrier.

### DEFINITIONS

As used in this disclosure, many organic compounds exist in “optically active forms”, i.e., they have the ability to rotate the lane of plane-polarized light. In describing an optically-active compound, the prefixes D and L or R and S are used to denote the absolute configuration of the molecule about its chiral center(s). The prefixes (+) and (−) or d and l are employed to designate the sign of rotation of plane-polarized light by the compound, with (−) or l meaning that the compound is levorotatory. A compound prefixed with (+) or d is dextrorotatory. For a given chemical structure, these compounds, called “stereoisomers”, are identical except that they are mirror images of one another. A specific stereoisomer may be referred to as an “enantiomer”, and a mixture of such isomers is often called an enantiomeric or racemic mixture. The characteristic feature of a chiral compound is the presence of an asymmetric carbon.

Stereochemical purity is of importance in the field of pharmaceuticals, where 16 of the 20 most prescribed drugs exhibit chirality. A case in point is provided by the L-form of the beta-adrenergic blocking agent propranolol, which is known to be 100 times more potent than the D-enantiomer. Furthermore, optical purity is important since certain isomers may actually be deleterious rather than simply inert. For example, it has been suggested that the D-enantiomer of thalidomide was a safe and effective sedative when prescribed for the control of morning sickness during pregnancy, while the corresponding L-enantiomer was a potent teratogen.

The statement made “(+)-enantiomer substantially free of the (−)-enantiomer” is meant to describe, for example, a compound that comprises 80% or more by weight of the (+)-enantiomer, and contains 20% or less by weight of the (−)-enantiomer, preferably is >90% by weight, preferably greater than 95% by weight, and even more preferred >99% by weight, based on the total weight of the active ingredient. By “(−)-enantiomer substantially free of the (+)-enantiomer” is meant to describe, for example, a compound that comprises >90% or more by weight of the (−)-enantiomer, and contains <10% by weight of the (+)-enantiomer, preferably greater than 95% by weight, and even more preferred >99% by weight of the (−)-enantiomer, based on the total weight of the active ingredient.

The use of the term “mammal” is meant to include any animal that may derive benefit from said treatment. Human and, non-human, companion species including the dog and cat are preferred.

The term “tolperisone-related compound” generally is meant to include tolperisone, its metabolites, its derivatives, or its salts and any isomers thereof. Eperisone and lanperisone are also contemplated.

### DETAILED DESCRIPTION OF THE INVENTION

The present invention relates to novel compositions and the use of a tolperisone-related compounds (including tolperisone, its metabolites, its derivatives, or its salts, and individual isomers thereof), for use of the treatment or prevention of disorders that arise from altered cell membrane excitability, including neuropathic pain, and pain associated with other nervous system disorders. These disorders include, but are not limited to, periodic paralyses and myotonias of several types, long QT syndrome, Brugada syndrome, malignant hyperthermia, myasthenia, epilepsy, ataxia, migraine, Alzheimer's Disease, Parkinson's Disease, schizophrenia, and hyperekplexia, painful diabetic neuropathy, postherpetic neuralgia, trigeminal neuralgia, complex regional pain syndrome, Guillain-Barre syndrome (GBS), Charcot-Marie-Tooth (CMT) disease, complex regional pain syndrome, type 1 (CRPS-1), ischemic neuropathy, fibromyalgia, chronic fatigue syndrome, painful spasticities, and other nervous system disorders that have pain as an attendant sign and/or symptom.

Tolperisone (2,4′-dimethyl-3-piperidino-propiophenone) is represented by the formula:

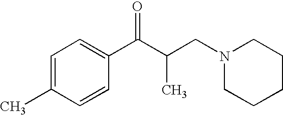

The racemic mixture of tolperisone has been marketed under trade names such as Mydeton®, Mydetone®, Muscalm®, Midocalm®, and Mydocalm® as a centrally acting muscle relaxant. It has been prescribed since 1959 for conditions in which skeletal muscle spasticity requires control, such as with low back spasm or spastic paralysis. The compound is an arylalkyl beta-aminoketone possessing an asymmetric carbon atom alpha to the carbonyl group. A number of pharmaceutical preparations have been described, including that in U.S. Pat. No. 6,500,455, which describes a racemate composition ranging from a 50/50 ratio to a 90/10 ratio of (−)-tolperisone to (+)-tolperisone. Other tolperisone-related compounds include eperisone and lanperisone.

Racemic tolperisone is reported to have tissue membrane stabilizing properties. The mechanism of action is hypothesized as a modulation of ionic channels, resulting in alterations of membrane permeability for sodium and potassium. See Hinck and Koppenhofer (Gen Physiol Biophys 2001 December; 20(4):413-29). Kocsis et al (Acta Pharm Hung 2002; 72(1):49-61) further describes the potential for tolperisone to inhibit voltage-gated sodium channels, contributing to membrane stability. Fels (Arch Pharm [Weinheim] 1996 April; 329(4):171-8) describes the potential for “lidocaine-like” activity of tolperisone through molecular modeling and conformational analyses. The author concludes that lidocaine and tolperisone, while different in their chemical structure, appear to share a common affinity for protein binding sites.

Keneko et al (Arch Int Pharmacodyn Ther 1987 June; 287(2):203-10) assessed the action of tolperisone and other compounds for their effects on spinal cord and descending neural pathways in rats. Tolperisone was found to depress segmental (limb) reflexes. Farkas et al (Neuropharmacology 1989 February; 28(2):161-73) studied the electrophysiological effects of tolperisone on spinal nerve root action potentials in cats. Tolperisone was found to dose-dependently inhibit both ventral nerve root reflexes and the dorsal nerve root reflex. Novales-Li et al (Eur J Pharmacol 1989 Sep. 22; 168(3):299-305) studied the effects of tolperisone on the calcium current of snail neurons. Tolperisone was found to inhibit calcium current dose-dependently.

The racemic compound tolperisone can be readily prepared by one of ordinary skill in the art. See Siozawa et al JP Appl. 86/239116, 1986 Eur. Pat. Appl EP 266577, 1988 [or Chem Abstr 109 P 128823g]. The entire disclosures are incorporated herein by reference.

In order to identify and isolate the (+) and (−) isomers of a compound such as that in Formula Z, the isomers must be resolved. This process may be achieved by a variety of means. The stereochemistry of tolperisone has been elucidated recently by Zsila and colleagues (Chirality 12:720-726; 2000) via circular dichroism and ultraviolet spectroscopy. As well, a stereoselective assay has been developed to quantify tolperisone enantiomers in plasma. See Yokoyama T et al (Chem Pharm Bull 40(1) 272-274 (1992)). Thus, the potential exists to both isolate the isomers, and also generate appreciable quantities of reasonably pure individual isomers. Each isomer is reported to have unique pharmacokinetic and pharmacodynamic properties in rat. Specifically, (+)-tolperisone demonstrates more muscle-relaxant properties, and (−)-tolperisone exhibits vasodilatory activity. See Furuta and Yoshikawa (Jpn J Pharmacol 1976; 26: 543-550). The isomers may be isolated, for example, by chiral chromatography.

Use of the enantiomers may confer advantages of use over the racemate, for example lesser adverse effects. The most serious adverse effect associated with racemic tolperisone is the incidence of anaphylaxis. See Ribi et al Swiss Med Weekly 2003; 133: 369-371. Allergic reactions to drugs represent some of the most commonly reported adverse events associated with drug therapy. Anaphylaxis is the most severe form of these allergic reactions. In addition, other frequently reported adverse effects associated with the use of racemic tolperisone include muscle weakness, muscle pain, headache, dizziness, and gastric complaints. See Pratzel et al Pain 67 1996; 417-25 for representative clinical trial results.

Thus, it may be desirable to find a compound with advantages of the racemic mixture of tolperisone, without the above-described disadvantages. In particular, there is a need for a compound which is effective for the treatment of pain and related disorders, without the above-described disadvantages and adverse effects associated with the administration of racemic tolperisone.

Thus, the present invention encompasses new compositions and methods for treating disorders that arise from altered cell membrane excitability, including neuropathic pain, and pain associated with other nervous system disorders in a mammal while reducing the concomitant liability of adverse effects associated with the administration of racemic tolperisone, which comprises administering to said mammal in need of treatment for pain, a therapeutically effective amount of a tolperisone-related compound, such as (+)-tolperisone, or (−)-tolperisone or a pharmaceutically acceptable salt thereof, substantially free of the undesired isomer, said amount being sufficient to alleviate pain, but insufficient to cause adverse effects associated with racemic tolperisone. The types of pain which may be treated according to methods of the present invention include, but are not limited to, acute pain, chronic pain (of both malignant and non-malignant sources), painful spasticities, neuropathic pain, migraine headache, persistent headache, tension headache, and other acute headache.

Any suitable route of administration can be employed for providing a mammal in need of treatment with a therapeutically or prophylactically effective dose of a tolperisone-related compound. For example, oral, mucosal (e.g., nasal, sublingual, buccal, rectal, vaginal), parenteral (e.g., intravenous, intramuscular), transdermal and subcutaneous routes can be employed. Desirably, the dosage range from about 1 mg to about 1000 mg of the active ingredient, preferably 10 mg to about 600 mg and more preferably from about 100 mg to about 300 mg of the active ingredient.

A pharmaceutical composition comprising a tolperisone-related molecule may optionally include high doses of magnesium (between about 2 to about 12 times the recommended daily dosage or 600 mgs to 5 gms elemental magnesium). See US Patent Publication No. 20050123626 which describes methods for administering high doses of magnesium, and herein incorporated by reference

Preferred pharmaceutical preparations may also have a means for controlled, delayed, sustained, extended release, or otherwise modified release of the active ingredient racemic tolperisone, (+)-tolperisone or (−)-tolperisone. U.S. Pat. No. 6,500,455 provides an example process by which a controlled-release preparation of tolperisone can be achieved. Specifically, this teaching is narrowed to racemate preparations that are of 50/50, 65/35, 70/30, 80/20, and 90/10 mixtures, with a preponderance of the (−)-tolperisone enantiomer.

Whether for unmodified or modified release preparations, racemic tolperisone and the optically pure isomers (+)-tolperisone and (−)-tolperisone may be prepared from pharmaceutically acceptable non-toxic acids including organic and non-organic acids. Such acids include maleic, acetic, benzene-sulfonic (besylate), benzoic, camphorsulfonic, citric, ethenesulfonic, fumaric, gluconic, glutamic, hydrobromaic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

A tolperisone-related compound can be combined as the active ingredient with a pharmaceutical carrier according to conventional pharmaceutical compounding techniques to form unmodified release compositions. The carrier may take a wide variety of forms depending on the form of preparation desired for administration, e.g., oral or parenteral (including intravenous injections or infusions). In preparing the compositions for oral dosage form, any of the usual pharmaceutical media may be employed, for example, water, glycols, oils, alcohols, flavoring agents, preservatives, coloring agents and the like in the case of oral liquid preparations, for example, suspensions, elixirs and solutions; or aerosols; or carriers such as starches, sugars, microcrystalline cellulose, stabilizers, diluents, granulating agents, lubricants, binders, fillers, disintegrating agents and the like in the case of oral solid preparations such as, powders, capsules and tablets, with the solid oral preparations being preferred over the liquid preparations. The preferred solid oral preparation is tablets. The most preferred solid oral preparation is coated tablets. Because of their ease of administration tablets and capsules represent the most advantageous oral dosage unit form, in which case solid pharmaceutical carriers are obviously employed. If desired, tablets may be coated by standard aqueous or nonaqueous techniques.

The pharmaceutical compositions of the present invention may also be formulated so as to provide modified or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations.

The controlled-release of the active ingredient may be stimulated by various inducers, for example pH, temperature, enzymes, water, or other physiological conditions or compounds. Various mechanisms of drug release exist. For example, in one embodiment, the controlled-release component may swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient (i.e., tolperisone) in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

Alternatively, the present invention relates to additional novel compositions and methods for treating or preventing disorders that arise from altered cell membrane excitability, including neuropathic pain, and pain associated with other nervous system disorders that entails administering a pharmaceutical composition comprising a compound of Formula Z and a pharmaceutically acceptable carrier to a subject. The compound includes one or more R1 or R2 or R3 moieties comprising s a hydride, a halogen, a hydroxyl, a lower alkoxy, lower alkyl, lower alkenyloxy, amino, lower-alkanoyl amino or lower alkylthio group, and the ring-structures contain carbon, amine or oxygen groups and have 4, 5, 6 or 7 carbon skeletons with single or double bonds and n=1-4.

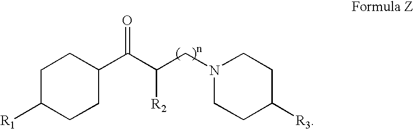

In order that this invention be more fully understood, the following examples are set forth. These examples are for the purpose of illustration only and are not to be construed as limiting the scope of the invention in any way.

The invention is demonstrated by administering a therapeutically relevant amount of racemic tolperisone, (+)-tolperisone or (−)-tolperisone to a mammal, using established models of experimental pain.

### EXAMPLE 1

### Chung Model of Peripheral Mononeuropathy

See the publication Chung J M and Kim S H, Pain 1992; 50: 355-63 for a full disclosure of study design. This technique is used to prepare a model of peripheral neuropathic pain. In the Chung model, the #5 and #6 lumbar nerves are ligated below the intervertebral foramen. This constriction injury causes as a distal perturbation in the pain signal pathways associated with the ipsilateral hind paw, modeling the perturbations evident in a peripheral neuropathic injury in a mammal. Specifically, the phenomenon of thermal hyperalgesia, cold allodynia, and tactile allodynia manifest themselves. The animals are challenged with thermal and mechanical stimuli to determine the degree of sensitivity in the target hindpaw post-surgically. Compounds that may be effective at treating neuropathic pain demonstrate an ability to decrease the pain sensitivity within the ipsilateral paw.

Tolperisone and its optically pure isomers are administered in one or more escalating doses. The doses are determined in a milligram of active ingredient per kilogram of animal weight scale. Tolperisone is administered to the animals to prevent the occurrence of pain in the peri-operative period, and/or to treat the pain resulting from the surgical intervention. Measurements are taken to determine how rapidly the animal withdraws the paw from a thermal or mechanical challenge. Comparisons can be made to negative and positive control group(s).

### EXAMPLE 2

### Model of Peripheral Polyneuropathy

See Ahlgren S C and Levine J D Brain Res 1993; 616: 171-175 for example use of the model. Subcutaneous injection of streptozotocin in rats induces a hyperglycemia and glucosuria phenomenon that manifests in a peripheral polyneuropathy. There is a resultant mechanical hyperalgesia and allodynia. This model is meant to represent diabetic peripheral neuropathy. The animals are challenged with thermal and mechanical stimuli to determine the sensitivity in the target hindpaw. Compounds that may be effective at treating neuropathic pain demonstrate an ability to decrease the pain sensitivity within the target paw. Comparisons can be made to negative and positive control group(s).

Tolperisone and its optically pure isomers are administered in one or more escalating doses. The doses are determined in a milligram of active ingredient per kilogram of animal weight scale. Tolperisone is given to the animals to prevent the occurrence of pain, and/or to treat the pain resulting from the in vivo chemical insult. Measurements are taken to determine how rapidly the animal withdraws the paw from a thermal or mechanical challenge. Comparisons can be made to negative and positive control group(s).

### EXAMPLE 3

### Modified-Release Preparation 1

Crystalline tolperisone hydrochloride with a grain size of 30 and 60 mesh is placed in a coating column operated with an air flow and is coated with a mixture of a polymer solution in chloroform with contains ethyl cellulose, hydroxypropyl cellulose and methanol. The coating solution is sprayed with 2.5 bar pressure into the column with a speed of 60 mL/min. The inlet temperature is roughly 60 degrees Celsius. After feed of the coating is ended, the tolperisone crystals which are dried quickly and which are coated with the polymer are removed from the coating column to the bottom.

### EXAMLE 4

### Modified-Release Preparation 2

This example teaches the preparation of an aqueous liquid suspension of tolperisone with delayed release. The aqueous vehicle is saturated with tolperisone and contains microencapsulated tolperisone suspended in water. Tolperisone is contained in the saturated aqueous solution in an amount which corresponds to its solubility. By administering a suspension of tolperisone-containing microcapsules in a tolperisone-saturated aqueous vehicle it is possible to make available tolperisone in a sufficient dose. This can be done by the tolperisone being made available in the form of suspended, tolperisone containing microcapsules and tolperisone as an aqueous solution in the mixing ratio which is required at the time. The amount of tolperisone in the microcapsules can be increased in order to take into account the amount of tolperisone solution replaced in the microcapsules.

The embodiments of the present invention described above are intended to be merely exemplary and those skilled in the art will recognize, or be able to ascertain using no more than routine experimentation, numerous equivalents to the specific procedures described herein. All such equivalents are considered to be within the scope of the present invention and are covered by the following claims. Other embodiments are within the following claims.

The contents of all references described herein are hereby incorporated by reference.

What is claimed:

1. A method for preventing or treating disorders that arise from altered cell membrane excitability, including neuropathic pain, and pain associated with other nervous system disorders in a subject in need of such treatment, said method comprising administering an effective amount of a pharmaceutical composition comprising a tolperisone-related compound and a pharmaceutically acceptable carrier to said subject.

2. The method of claim 1, wherein the compound is racemic tolperisone.

3. The method of claim 1, wherein the compound is (+)-tolperisone, substantially free of the corresponding (−)-isomer.

4. The method of claim 1, wherein the compound is (−)-tolperisone, substantially free of the corresponding (+)-isomer.

5. The method of claim 1, wherein the compound is administered orally.

6. The method of claim 1, wherein the compound is administered parenterally.

7. The method of claim 1, wherein the compound is administered by a transdermal route.

8. The method of claim 1, wherein the pharmaceutical composition is an unmodified release pharmaceutical composition.

9. The method of claim 1, wherein the pharmaceutical composition is a modified release pharmaceutical composition.

10. The method of claim 1, wherein said nervous system disorder is selected from the group consisting of periodic paralyses and myotonias of several types, long QT syndrome, Brugada syndrome, malignant hyperthermia, myasthenia, epilepsy, ataxia, migraine, Alzheimer's Disease, Parkinson's Disease, schizophrenia, and hyperekplexia, painful diabetic neuropathy, postherpetic neuralgia, trigeminal neuralgia, complex regional pain syndrome, Guillain-Barre syndrome (GBS), Charcot-Marie-Tooth (CMT) disease, complex regional pain syndrome, type 1 (CRPS-1), ischemic neuropathy, fibromyalgia, chronic fatigue syndrome and painful spasticity.

11. The method of claim 1, wherein the pharmaceutical composition further comprises a high dose of magnesium.

12. A pharmaceutical composition comprising (+)-tolperisone, substantially free of the corresponding (−)-isomer, and a pharmaceutically effective carrier.

13. A pharmaceutical composition comprising (−)-tolperisone, substantially free of the corresponding (+)-isomer, and a pharmaceutically effective carrier.

14. A method for preventing or disorders that arise from altered cell membrane excitability, including neuropathic pain, and pain associated with other nervous system disorders in a subject in need of such treatment, said method comprising administering an effective amount of a pharmaceutical composition comprising a compound of Formula Z and a pharmaceutically acceptable carrier to said subject: 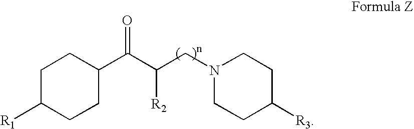
wherein the compound includes one or more R1 or R2 or R3 moieties comprising s a hydride, a halogen, a hydroxyl, a lower alkoxy, lower alkyl, lower alkenyloxy, amino, lower-alkanoyl amino or lower alkylthio group, and the ring-structures contain carbon, amine or oxygen groups and have 4, 5, 6 or 7 carbon skeletons with single or double bonds and n=1-4.

15. The method of claim 14, wherein the compound is racemic tolperisone.

16. The method of claim 14, wherein the compound is (+)-tolperisone, substantially free of the corresponding (−)-isomer.

17. The method of claim 14, wherein the compound is (−)-tolperisone, substantially free of the corresponding (+)-isomer.

18. The method of claim 14, wherein the compound is administered orally.

19. The method of claim 14, wherein the compound is administered parenterally.

20. The method of claim 14, wherein the compound is administered by a transdermal route.

21. The method of claim 14, wherein the pharmaceutical composition further comprises a high dose of magnesium.

22. The method of claim 14, wherein said nervous system disorder is selected from the group consisting of periodic paralyses and myotonias of several types, long QT syndrome, Brugada syndrome, malignant hyperthermia, myasthenia, epilepsy, ataxia, migraine, Alzheimer's Disease, Parkinson's Disease, schizophrenia, and hyperekplexia, painful diabetic neuropathy, postherpetic neuralgia, trigeminal neuralgia, complex regional pain syndrome, Guillain-Barre syndrome (GBS), Charcot-Marie-Tooth (CMT) disease, complex regional pain syndrome, type 1 (CRPS-1), ischemic neuropathy, fibromyalgia, chronic fatigue syndrome and painful spasticity.
